(19) United States
(12) Patent Application Publication
(10) Pub. No.: US 2005/0038371 A1
(43) Pub. Date: Feb. 17, 2005

(54) Controlled cerebrospinal infusion and shunt system

(76) Inventors: Sanford Reich, Providence, RI (US); James E Sluetz, N. Attleboro, MA (US)

Correspondence Address: NIELDS & LEMACK, 176 EAST MAIN STREET, SUITE 7, WESTBORO, MA, 01581, US

(21) Appl. No.: 10/503,607

(22) PCT Filed: Feb. 12, 2003
(86) PCT No.: PCT/US03/04099

Related U.S. Application Data
(60) Provisional application No. 60/356,398, filed on Feb. 13, 2002.
(60) Provisional application No. 60/358,648, filed on Feb. 21, 2002.

(51) Int. Cl. A61M 5/00

(52) U.S. Cl. 604/9

(57) ABSTRACT

An implantable, battery-operated controlled cerebral infusion and shunt (CCIS) system and method that is microprocessor controlled via algorithms stored in its memory. The system includes a programmable infusion system and a multi mode drainage system that contains at least two flow paths: a low resistance flow path for when the patient is in the supine or substantially supine position and a flow path containing a programmable variable check valve to prevent over-drainage when the patient is in the upright or substantially upright position. The combination of the above two functions allows modulation of the cerebrospinal fluid (CSF) turnover rate.

Priority is claimed of Provisional Appln. Ser. Nos. 60/356,398 filed Feb. 13, 2002 and 60/358,648 filed Feb. 21, 2002, the disclosures of which are incorporated by reference.

### BACKGROUND OF THE INVENTION

The human skull is primarily occupied by brain tissue and the supporting blood vessels. About ten percent of this volume is clear fluid with small amounts of dissolved protein, sugar and salts. This cerebrospinal fluid (CSF) cushions the delicate brain and spinal cord tissues from injuries and maintains the proper balance of nutrients and salts around the central nervous system.

A system of four interconnecting cavities, known as ventricles, in the brain provide pathways through which the CSF circulates from deep within the brain, around the spinal column, and over the surfaces of the brain. CSF is continually being created. In fact, about three to five times the volume contained in the skull at any point in time is produced on a daily basis.

Normally, almost all of the CSF is absorbed into the bloodstream, thus maintaining the delicate balance between CSF production and absorption. Normal intraventricular pressure (IVP) is 10 mm Hg (patient in a horizontal position) and typically varies from 5 to 15 mm Hg. Above 20 mm Hg for any sustained period can lead to serious complications. Normal IVP is between −5 mm Hg and 0 mm Hg when the patient is in the upright position.

For patients with normal CSF generation and normal CSF absorption, it may be beneficial to enhance the cerebrospinal turnover rate. The increased CSF turnover rate will remove toxins that may be present in the CSF. Increased cerebrospinal turnover rate may also be of benefit if excess therapeutic drug concentration build-up in the CSF is a concern. The use of shunt device together with lavage solution (saline solution with or without amenable drugs) infusion more effectively modulates the cerebrospinal turnover rate.

This combination therapy may be used to treat a range of neurological conditions that may include adult-onset dementia of the Alzheimer's type, Parkinson's disease, a cancerous tumor growth involving the brain, or any combination of these or other drug amenable neurological conditions.

The introduction of two cannulae into the brain area through the same bore hole allows for optimal placement of an infusion cannula for providing lavage solution infusion and CSF drainage cannula to modulate the CSF turnover rate. The CSF drainage is controlled via adjustments to the programmable IVP range. The amount of CSF drained by the shunt from the ventricles is a function of the IVP because the drained CSF flow is a function of the shunt pressure difference (the pressure difference between the proximal IVP and the distal shunt pressure). The amount of CSF generated by the choroid plexus is also influenced by the IVP because the CSF transport through the choroid plexus is a function of the cerebral perfusion pressure (the pressure difference between the mean arterial pressure and the mean IVP). For a patient with normal or somewhat compromised CSF absorption capability, the selection of a particular range for controlling the IVP will influence the amount of generated CSF and result in influencing the amount of CSF that is drained. In these cases, the increase in CSF fluid that bathes the surfaces of the brain will increase the wash out of the CSF within the brain. Thus, controlling the IVP and the lavage solution infusion time profile will result in changing the CSF turnover rate relative to specific localized areas of the brain and result in the ability to modulate the CSF turnover rate.

However, when providing CSF turnover rate therapy into patients with significantly decreased cerebrospinal fluid production and decreased cerebrospinal fluid absorption, it is important to avoid either under-drainage or over-drainage.

Shunt installation is a surgical procedure in which a valve system is usually implanted while the patient is under general anesthesia. In this commonly used procedure for hydrocephalus, a small hole is made in the skull and the protective membrane overlaying the brain. An incision is made in the abdomen and the valve unit and associated tubing are introduced under the skin between the scalp and the abdominal incisions. Usually one ventricular cannula is inserted into the lateral ventricle and connected to the drainage tube, which is inserted in the abdominal cavity. This system is intended to allow CSF from the ventricle to travel through the implanted tubes into the abdominal cavity, where it is then absorbed into the bloodstream.

CSF turnover rate is modulated by the infusion of a lavage solution and the drainage through the shunt. Under-drainage occurs when CSF is not removed quickly enough relative to the CSF generation and lavage infusion. Over-drainage occurs when the shunt allows CSF to drain from the ventricles more quickly relative to CSF generation and lavage infusion.

The shunt drainage rate of the shunts varies depending on the patient's relative position. In an upright position, an increased rate of CSF flow is generated, since gravity serves to create siphoning pressure, which will aid in the drainage process. In the supine, or horizontal, position, drainage is caused solely by the imbalance of pressure. Current shunt therapy devices are not designed to effectively treat over-drainage. These devices still maintain a large negative IVP (over-drainage) when the patient is in the upright position. A change of valve to a higher pressure cannot be relied upon to cure it, though it appears to do so in some cases. Anti-siphon devices, which consist of a small button inserted into the shunt tubing, may sometimes solve the problem. Some shunts have these built-in, but neurological opinion varies as to whether they should be used. To change a valve pressure, surgery is necessary to remove the valve and insert another. A relatively new shunt, the ‘programmable’ or adjustable shunt, is intended to allow adjustment of the working pressure of the valve without surgery. This valve contains magnets that allow the valve pressure setting to be altered by a transcutaneous magnetic field placed over the scalp. This is useful where the need for a valve of a different pressure arises, but the adjustable valve is no less prone to the over-drainage issue than any other and it cannot be used to treat this condition.

All the current passive shunts in clinical use rely on a precarious equilibrium between under-drainage in a lying position and acceptable over-drainage in an upright position. Some shunts use a variable resistance element to control drainage rate when the patient is standing. Other shunts use a programmable check valve for control of under-drainage along with a flow resistance element to limit the flow rate during over-drainage when the patient is in the standing position.

This CCIS invention, designed for chronic ambulatory therapy, describes a solution infusion capability via a programmable infusion device with an integral active shunt device that controls and monitors the intracranial pressure for all patient positions based on feedback from sensors. The CCIS is used to modulate the CSF turnover rate.

### SUMMARY OF THE INVENTION

The limitations of the current intracranial therapy, i.e., using only a shunt, have been overcome by the present invention. This invention is directed to an active implantable device that combines two functions: cerebrospinal solution (preferably lavage solution) infusion using a programmable infusion pump, and cerebrospinal fluid (CSF) shunting using a programmable shunt. There are two important benefits to be had by combining these two functions into one device. First, direct cerebrospinal lavage solution infusion is often needed to enhance the CSF turnover rate. Second, controlled clearance of CSF provides a more controllable physiological sink for the CSF especially for CSF toxins.

The programmable infusion pump is a constant pressure flow-limited design whose flow output is programmable with flow modulation provided by an infusion bi-stable latching valve and monitored by a pressure sensor.

The shunt system is a multi-mode drainage system that contains at least two flow paths: a low resistance flow path for when the patient is in the supine or substantially supine position and a flow path containing a programmable variable check valve to prevent over-drainage when the patient is in the upright or substantially upright position. By providing at least two flow paths, the IVP pressure can be controlled within a programmed physiological range. This shunting of CSF fluid also has the benefit of providing a means for improving the CSF turnover rate.

This CCIS System, together with related non-invasive diagnostic algorithms, comprises a dual therapy cerebrospinal infusion and shunt management system.

### BRIEF DESCRIPTION OF THE DRAWINGS

FIG. 1 is a flow schematic of the CCIS System;

FIG. 2 is a view of the first embodiment of the variable check valve, located within the CCIS System;

FIG. 3 is a graph demonstrating the check valve's performance over a range of conditions;

FIG. 4 is the preferred implantation of the CSF system in the patient; and

FIG. 5 is a second embodiment of the variable pressure valve.

### DETAILED DESCRIPTION OF THE INVENTION

The CCIS device is an implantable active battery operated device that is microprocessor controlled via algorithms stored in its memory. The CCIS device is a dual therapy system containing a programmable solution infusion device that is integral with a programmable actively controlled shunt system. The CCIS device can be implanted in the abdomen with an attached dual lumen cannula used for CSF infusion and for CSF shunting. A second shorter cannula attached to the CCIS device is used to divert the shunted CSF fluid into a suitable location, such as the peritoneal cavity in the abdomen.

In the preferred embodiment, the CCIS device contains a programmable lavage solution infusion system. A lavage solution reservoir is preferably within a pressurized container such that at body temperature, it produces a positive pressure. A refill septum can be provided, such as on the top surface of the device, so that the reservoir can be easily refilled, for example via a transcutaneous needle attached to a refill syringe. A flow restrictor, preferably consisting of a capillary tube of glass, is used to limit the maximum flow rate to the range of shunt drainage flow rates, for example, less than 5 ml per hour. A pressure sensor, preferably an absolute pressure sensor, is located in the flow path downstream of the flow restrictor and is preferably used to calculate the flow rate of the infused lavage solution. Downstream of the pressure sensor is a suitable device to control the flow of the infused lavage solution, preferably an infusion bi-stable latching valve that gates the flow between an on position and an off position. The control of the infusion bi-stable latching valve position is typically performed by the microprocessor and its interface electronics. The lavage solution infusion volumes and delivery profiles are thus controlled by the time profiles and the time durations of the programmed flow periods. This approach allows the infusion system to provide a very wide range of volumes and delivery profiles.

The actual flow rate and lavage solution volume delivery during the infusion bi-stable valve “on” flow period can be determined using the pressure sensor and the following algorithm. The flow determination algorithm is based on measuring the pressure on the upstream and downstream side of the flow restrictor with a known flow resistance. This flow resistance is determined during manufacture of the flow restrictor and is a function of the resistance or viscosity of the solution flowing through it. The upstream pressure can be measured just prior to the opening of the infusion bi-stable latching valve. Since the valve is not open, the pressure on both ends of the flow restrictor will be equal and will be equivalent to the upstream pressure. The downstream pressure is measured after the infusion bi-stable latching valve is opened. The pressure differential across the flow restrictor is the difference between the two pressure readings. The flow rate is determined by dividing the pressure differential by the known flow resistance of the solution. The solution volume delivered during each flow period is simply the flow rate multiplied by the infusion bi-stable latching valve “on” time.

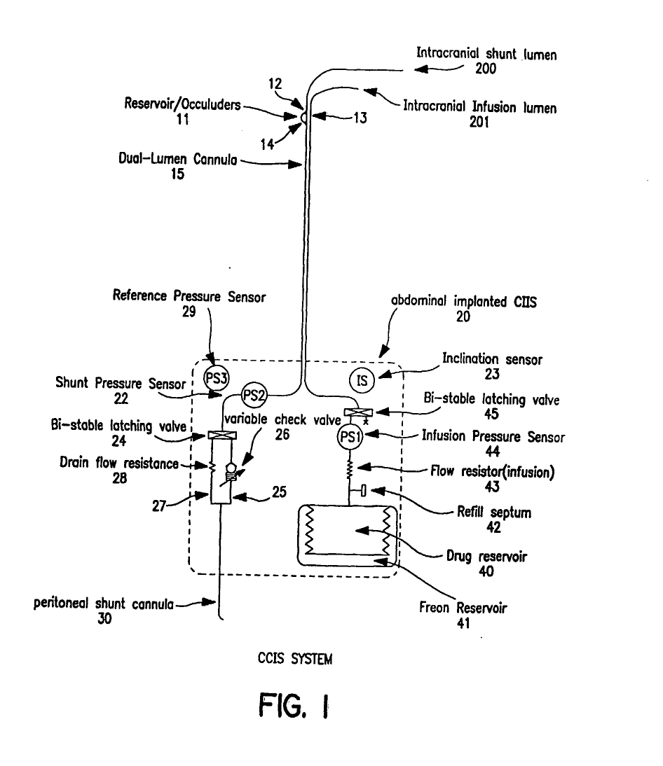

The CCIS system also contains a dual mode shunt system. The programmable shunt contains at least two flow paths: (1) a supine mode: a low resistance flow path for when the patient is in the supine or substantially supine position and (2) an upright mode: a flow path containing a programmable variable check valve to prevent over-drainage when the patient is in the upright or substantially upright position. A shunt bi-stable latching valve directs the CSF flow to either the low resistance path or the check valve path based on an inclination sensor within the CCIS device. If the inclination sensor angle is below a programmable critical angle, the shunt bi-stable latching valve directs flow to the low resistance path. If the inclination sensor angle is equal to or above a critical programmable angle, the shunt bi-stable latching valve directs flow to the check valve path. For purposes of illustration, a dual mode device will be described; however, the present invention is not limited to only two modes. FIG. 1 shows the flow schematic of the CCIS System.

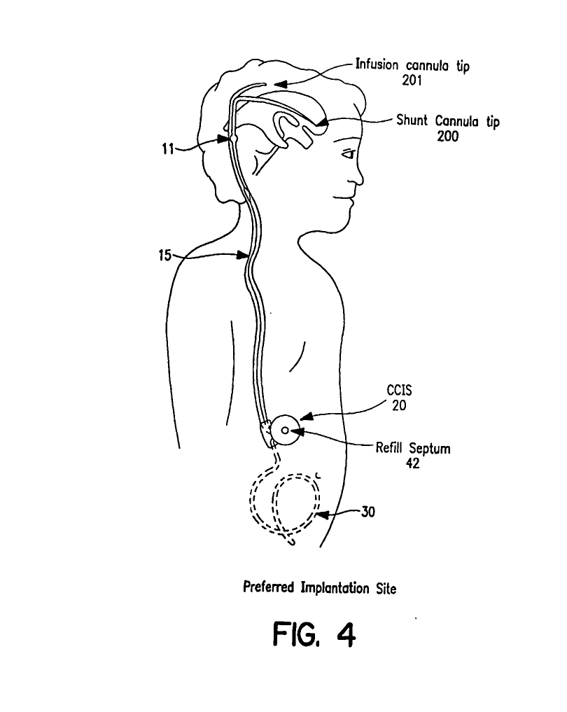

Turning now to FIG. 4, the implant location of the proximal tip of the intracranial infusion lumen 201 is determined by the physician to maximize the lavage solution effectiveness. It serves to supply infused medication to the cranial area. The infusion lumen is in fluid communication with one branch tube of the dual-lumen cannula 15. The cannula 15 traverses the body from the cranial area to the implanted Controlled Cerebral Infusion and Shunt (CCIS) System device 20. As shown in FIG. 1, the lavage solution reservoir 40 is typically a positive pressure device. In the preferred embodiment, a freon (or other compressible fluid) reservoir 41 envelops a pressurized bellows (preferably titanium), which comprises the lavage solution reservoir 40. At body temperature, the freon expands and produces a positive pressure on the lavage solution reservoir 40. Bellows fluid reservoirs are standard and commonplace in the art because the gas pressure not only propels the fluid but also provides for make-up volume. A refill septum 42 is typically provided in a suitable location, such as on the top surface of the device, so that the reservoir can be refilled, for example via a transcutaneous needle attached to a refill syringe. A flow restrictor 43, preferably consisting of a capillary tube of glass, can be used to limit the maximum flow rate to the range of shunt drainage flow rates, e.g., less than 5 ml per hour, or to some other value. An absolute pressure sensor, known as the infusion pressure sensor 44, can be located in the flow path, preferably downstream of the flow restrictor. The infusion pressure sensor is preferably a MEMS (Micro-Electro-Mechanical Systems) absolute pressure sensing silicon element. Downstream of the pressure sensor is an infusion bi-stable latching valve 45 that gates the flow between on and off. The control of the bi-stable latching valve position is performed by an implanted microprocessor and its interface electronics. The fluid infusion volumes and delivery profiles are thus controlled by the time profiles and the time durations of the programmed flow periods.

The actual flow rate and fluid volume delivery during the infusion bi-stable valve 45 “on” flow period may be determined using the infusion pressure sensor 44. The flow determination algorithm is based on measuring the pressure on the upstream and downstream side of the flow restrictor 43 with a known flow resistance. The flow resistance of the flow restrictor 43 can be determined during manufacture and is a function of the viscosity of the fluid used. The upstream pressure is measured just prior to the opening of the infusion bi-stable latching valve 45. The downstream pressure is measured after the infusion bi-stable latching valve is opened. The pressure differential across the flow restrictor is the difference between the two pressure readings. The flow rate is determined by dividing this pressure differential by the previously known flow resistance of the fluid. Multiplying the flow rate by the infusion bi-stable latching valve 45 “on” time approximates the fluid volume delivered during each flow period.

The infusion pressure sensor 44 also serves as a diagnostic tool for detecting flow blockages in the infusion path. For example, if the infusion pressure sensor 44 measures the same pressure when the infusion bi-stable latching valve 45 is in the “on” position as when it is in the “off” position, a blockage may have occurred.

The combination of the infusion pressure sensor 44, the infusion bi-stable latching valve 45, and the flow restrictor 43 allows very accurate measurements of flow rate and therefore volume. This degree of accuracy allows this infusion system to be viable in this application.

The intracranial shunt lumen 200 is typically implanted in the ventricle of the patient's brain. It serves as the source for the CSF fluid into the CCIS system. The ventricular cannula is in fluid communication with the reservoir/occluders device 11. This device is implanted just beneath the scalp and can be actuated by pressing on the scalp. This device contains a reservoir 13 for holding CSF fluid. On either side of the reservoir is a manual blocking mechanism, known as an occluder. One nearer to the ventricle is known as the proximal occluder 12, while the other is the distal occluder 14. These occluders allow the physician to interrupt the flow of CSF to perform a number of in-office non-invasive diagnostics.

The distal occluder 14 is in fluid communication with one branch of the dual lumen cannula 15, which is in fluid communication with the Controlled Cerebrospinal Infusion and Shunt (CCIS) device 20. The CCIS device is preferably located in the subcutaneous abdominal area. It regulates the flow of CSF through it, and the outgoing CSF flows into the peritoneal shunt cannula 30. This outlet cannula is implanted such that its distal (far) end is inserted into a suitable drainage area, such as the peritoneal cavity.

FIG. 4 shows the preferred implantation site for the CCIS system. The proximal end of the dual-lumen inlet cannula 15 separates into multiple, preferably two, separate cannulas that are implanted within the intracranial space. The implant location of the fluid infusion proximal cannula tip 201 can be determined by the physician to maximize the fluid effectiveness. The implant location of the shunt proximal cannula tip 200 is typically in the lateral ventricles that are near the choroids plexus. The inlet shunt cannula is preferably in fluid communications with an inline reservoir 11 with manual occluders at its proximal ana distal ends. The purpose of this component is to assist in non-invasive diagnostic algorithms that are described in co-pending PCT International Application No. PCT/US03/0095 entitled “Diagnostic Algorithms for a CSF Physiologic Controller”, the disclosure of which is hereby incorporated by reference. This inline reservoir component 11 is typically implanted under the scalp and is in fluid communications with the dual-lumen cannula 15. The dual-lumen cannula 15 traverses the body from the scalp to the CCIS device 20, which is preferably located in the subcutaneous area of the abdominal cavity. The outlet shunt cannula 30 is implanted such that its distal end is inserted in a suitable drainage area, such as the peritoneal region. The proximal end of the outlet cannula is attached to the outlet connector of the CCIS device 20. Refill septum 210 is typically implanted in such a position to allow it to be easily refilled, such as via a transcutaneous needle attached to a refill syringe.

Referring back to FIG. 1, the CCIS System 20 contains all of the mechanisms required to implement the CSF flow.

The dual-lumen cannula 15 flows into the shunt pressure sensor component 22, located within the CCIS device 20. The purpose of this sensor is to determine the relative pressure of CSF at the shunt branch of the dual-lumen cannula 15. The following is for illustrative purposes only; a number of different embodiments could be used to implement the pressure sensor. In this embodiment, the pressure sensor component 22 is a MEMS (Micro-Electro-Mechanical Systems) absolute pressure sensing silicon elements. A second, reference pressure sensor 29, of the same type, is also used to determine the actual CSF pressure at the inlet. The two MEMS silicon pressure-sensing elements may be attached to a common vacuum. The non-vacuum sides of each are oil-coupled to the force-collecting diaphragms. The top force-collecting diaphragm is integral with a flat portion of the CSF fluid path and measures the absolute pressure in the CSF path. This corresponds to shunt pressure sensor 22. The lower force-collecting diaphragm is in communication with the outside bottom portion of the device and measures the absolute pressure on the outside of the device. This outside pressure sensing element, or reference pressure sensor 29, measures the tissue pressure of the implanted device and closely tracks the atmospheric pressure. A mechanical guard over the outside force-collecting diaphragm protects it from mechanical forces that may produce pressure artifacts. The difference between the two absolute pressure sensors is the gauge pressure of the CSF at the inlet to the CCIS device. This pressure is indicative of the intraventricular pressure (IVP). In the supine position, this reading is roughly equivalent to the IVP. In the upright position, this reading is the IVP plus the siphon pressure created by the shunt. By using the inclination sensor, it is possible to determine the actual IVP of the patient regardless of the inclination angle. In normal operation, the pressure sensor monitors the intraventricular pressure (IVP) not continuously, but periodically, for example, every 2-5 minutes. These readings can be stored in the device's memory. Using the telemetry capability of the CCIS device to download the information to the external programmer, the physician may review daily changes in IVP to diagnostic purposes. For example, the physician may choose to do this when a patient complains of headaches. The CCIS device can sample the pressure sensor at any time to determine the IVP, as measured at the input to the device.

The inclination sensor 23 is a gravity-detecting sensor that is used to determine the patient's inclination angle. It is used to control the multi mode CSF shunt system. This sensor also detects patient activity, such as when the patient is resting or moving about. Both the inclination and activity functions may be utilized to control the shunt bi-stable latching valve 24.

The shunt bi-stable latching valve 24 directs the CSF flow to the low resistance, supine mode path 27 when the inclination sensor 23 indicates that the patient is in a supine or substantially supine position; or the upright mode flow path 25 when the inclination sensor indicates that the patient is in an upright position.

The supine mode flow path 27 includes a supine flow resistance 28, which is designed to prevent against under-drainage and keep the IVP within the normal upper limit of 15 mm Hg. In this embodiment, the supine flow resistance is simply the resistance of the cannula in the supine mode flow path. The upright mode flow path 25 provides a variable high resistance flow path that is designed to prevent over-drainage. The variable high resistance flow path is provided by a variable check valve 26 whose cracking pressure is automatically adjusted based on the inclination angle.

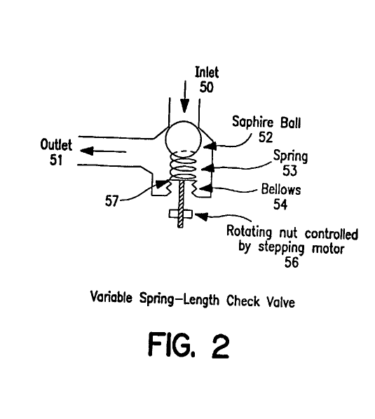

FIG. 2 shows a suitable design for a variable check valve. This diagram is for illustrative purposes only, and the check valve is not limited to a particular valve embodiment. The CSF flow originates at the inlet 50. A ball 52 serves to block the CSF from passing from the inlet 50 to the outlet 51. The ball 52 is preferably constructed of a material not deleterious to the application, such as sapphire, which does not interact with the cerebrospinal fluid. The ball is preferably small in diameter in order to ensure the best seal when the ball is resting on the inlet 50. For CSF to pass to the outlet, the pressure of the CSF at the inlet 50 must exceed the pressure exerted by the spring 53. The point at which this occurs is known as the cracking pressure. At this point, the ball will rise and allow the CSF to flow through the inlet 50 and onto the outlet. The spring 53 is located between the sapphire ball 52 and a horizontal platform 57. This horizontal platform can be moved both up and down by rotating screw 56. As the horizontal platform is moved up, the cracking force increases. Likewise, as the horizontal platform is lowered, the cracking force decreases. Bellows 54 covers the horizontal platform to insure that the valve is fluid-tight. The rotating screw 56 is controlled by a nut 55, which in turn is controlled by a stepping motor (not shown). In this embodiment, the stepping motor controls the nut as a function of the patient's inclination angle, as determined by the inclination sensor 23.

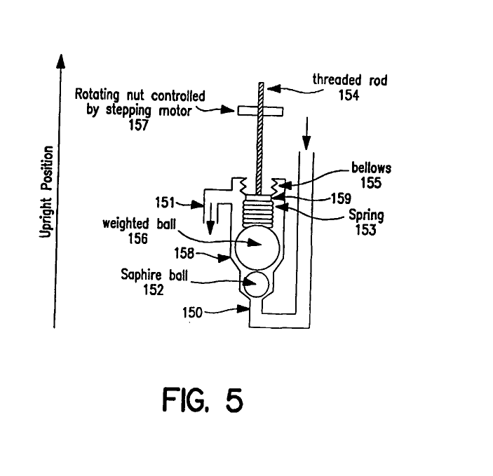

FIG. 5 shows a second, preferred embodiment of the variable check valve assembly. The CSF flow originates at the inlet 150 at the base of the valve. A small ball 152, again preferably sapphire, sits atop the inlet 150, forming a seal. Sapphire is used because it does not interact with the CSF. The ball is preferably small in diameter in order to ensure the best seal when the ball is resting on the inlet 150. This ball, which is held in place by valve housing 158, serves to block the CSF from passing from the inlet 150 to the outlet 151. A weighted ball 156, preferably made of tantalum because of its high density and its inertness, is located between the sapphire ball 152 and the spring 153 and rests against the valve housing 158. In this illustration, the weighted ball is shown to be larger than the sapphire ball. While this is the preferred implementation, the invention is not subject to this limitation. In order for the sapphire ball to be unseated, the pressure of the CSF at the inlet 50 must exceed the pressure exerted by the spring 153 plus the downward force of the weighted ball 156. The point at which this occurs is known as the cracking pressure. Note that when the patient is in the upright position, the downward force of the weighted ball 156 on the sapphire ball is equal to its weight. However, in the vertical position, the weighted ball exerts no additional force on the sapphire ball, as the gravitational force will be against the valve housing 158. Thus the force exerted by the weighted ball 156 on the sapphire ball can be expressed as the weight of the ball multiplied by the sine of the inclination angle of the patient, where an inclination angle of 0° signifies a supine position and an inclinarion angle of 90° indicates a fully upright position. The spring 153 is located between the sapphire ball 152 and a horizontal platform 159. This horizontal platform can be moved both up and down by rotating threaded rod 154. As the horizontal platform 159 is moved toward the sapphire ball 152, the force of the spring 153 increases, therefore the cracking force increases. Likewise, as the horizontal platform 159 is moved away from the sapphire ball, the force of the spring decreases, therefore the cracking force decreases. Bellows 155 covers the horizontal platform 159 and seals to the valve housing 158 to insure that the valve is fluid-tight. The threaded rod 154 is controlled by a rotating nut 157, which in turn is controlled by a stepping motor (not shown). The stepping motor is controlled by the microprocessor in the device. In this embodiment, the stepping motor controls the nut, which turns the threaded rod, and causes the horizontal platform to move. This adjustment is carried out to set the correct cracking pressure when the patient is in the upright position. The cracking pressure is made up of two components, a fixed component, which is set using the spring force and a gravitational variable component, which is determined by the weighted ball. The variation in cracking pressure required as the patient changes inclination are mostly handled by the variation in the downward gravitational force of the weighted ball, thereby significantly reducing the power required to maintain a stable intraventricular pressure over a range of inclination angles.

Those skilled in the art will appreciate that the gravitational component of the valve assembly could be in fluid communication with a separate inlet from the inlet that the fixed component is in fluid communication with, in which case the gravitational component and fixed component would function in series.

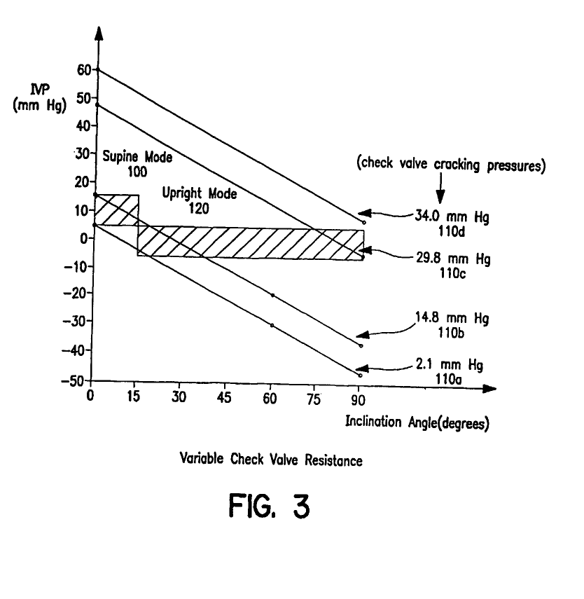

FIG. 3 graphically illustrates the operation of the check valve. This particular graph is provided for purposes of illustration, and the invention is not limited to this functionality. This graph shows intraventricular pressure graphed as the vertical axis, with patient's inclination angle as the horizontal axis. For clarity, 0 degrees denotes a person in the completely horizontal position, while 90 degrees is a patient in the fully upright position. In this example, the siphon length was 62 cm. Four diagonal lines 110a-d show lines of constant check valve cracking pressure. As an example, if the check valve cracking pressure were held constant at 2.1 mm Hg, as in line 110a, the IVP would be 5 mm Hg in the supine position. The IVP would decrease as the inclination angle increased, reaching a value of about −45 mm Hg when the patient is fully upright. Similarly, line 110d illustrates that for a cracking pressure of 34.0 mm Hg, the IVP is 60 mm Hg when the patient is fully supine and about 10 mm Hg when the patient is completely upright. Lines 110b and 110c show similar trends at 14.8 mm and 29.8 mm, respectively. The shaded area, supine mode 100, denotes the desired IVP when the patient is in the supine or substantially supine position. As used herein, substantially supine is defined as less than about 15 degrees of inclination (accordingly, substantially upright is an inclination angle greater than about 15°). In the present invention, this result is achieved using the low resistance supine mode flow path 27 in the CSF. Once the patient's inclination angle exceeds about 15 degrees, the CSF uses the upright mode flow path 25. In this mode, the desired IVP range is shown in the shaded area, upright mode 120. At 15 degrees, the valve cracking pressure is between 2.1 mm and 14.8 mm in order to achieve an IVP of −5 to 5 mm Hg. As the patient becomes more upright (i.e., the inclination angle approaches 90°), the cracking pressure increases in order to maintain the desired IVP. When the patient is fully upright, the cracking pressure is about 29.8 mm Hg in order to maintain the proper IVP.

These graphs can be generated using the preferred embodiment of the programmable cracking pressure valve described in FIG. 5. While the patient is in the upright position, the spring tension is adjusted such that the IVP is between 5 and −5 mm Hg. As the patient reclines toward horizontal, a lower cracking pressure is needed to maintain the desired IVP range. The gravitational component of the cracking pressure, which is contributed by the weighted ball, is reduced as the patient reclines, thereby lowering, without any use of battery power, the cracking pressure of the valve. In this way, the siphon pressure created by the fluid contained within the length of the inlet cannula from the brain to the device is roughly counterbalanced by the effect of the weighted ball. By using the combination of the programmable spring force and the weighted ball, it is therefore possible to maintain the IVP within the desired range as shown in FIG. 3.

In addition to the elements described above, which are part of the flow paths, there is a microprocessor-based subsystem internal to the CCIS device. This subsystem preferably comprises a microprocessor, its associated memory, a Real Time Clock, a wireless transceiver and other essential electronics. An internal battery powers this subsystem. The microprocessor is responsible for monitoring and controlling many of the operations enumerated above, such as monitoring the inclination sensor, adjusting the check valve cracking pressure in response to changes in inclination, monitoring the pressure sensor, and controlling the infusion bi-stable latching valve. The microprocessor is also capable of receiving commands and returning status to the external programmer via the wireless transceiver. The memory is used to store data requested by the external programmer, such as pressure readings, inclination angle, and time. These data can be transmitted back to the external programmer as requested, via the wireless transceiver. The Real Time Clock is used to enable the device to perform certain diagnostics at specific times.

In conjunction with the CCIS device, there is an accompanying external programmer. This programmer is typically used by a physician, and is used to program critical parameters in the CCIS device, retrieve stored information from the device, and perform other types of communication with the CCIS device. The external programmer can also be used to perform a number of diagnostic procedures in conjunction with the CCIS device. The external programmer permits the physician to program the desired critical angle at which the CCIS device switches from supine to upright mode. The external programmer can also be used to preset the spring tension for the preferred embodiment of the variable cracking pressure valve, shown in FIG. 5. This adjustment is used to create the patient unique version of FIG. 3. The external programmer also contains a MEMS based barometer that is used to calibrate the tissue pressure sensor in the CCIS device.

The lavage solution infusion daily time profiles are determined by the flow initiation times and the flow duration times during each 24-hour cycle. Using an accompanying programmer, the physician may program an average daily infusion volume to be distributed in a number of ways, such as in equal bolus volumes over each 24-hour period, or in a customized time profile over each 24-hour period.

The external programmer can take many different physical forms. It preferably comprises the following set of components:
a processor unit to perform the necessary algorithms and calculations;
an internal memory to store data received from the device, and other relevant information;
a data input device to accept input from the physician;
a data output device to display data to the physician; and a communication port to transmit information to the CCIS device.

The external programmer can be a custom developed apparatus, or can be an existing device, such as a Palm™ handheld or laptop computer. In the scenario where a Palm™ handheld is used, the criteria above are met as follows. The processor unit and internal memory are standard elements of the Palm™ handheld. The data input device is the touch screen of the device, or the optional keyboard. The data output device is also the touch screen. Lastly, the communication to the CCIS device is performed by an optional wireless module that can be connected to the Palm™ handheld.

1. A system for precisely regulating the flow of a solution from a reservoir, comprising:
a flow restrictor downstream from said reservoir, in fluid communication with said reservoir, said flow restrictor having an output;
a pressure sensor downstream from said flow restrictor, said sensor adapted to measure fluid pressure at said output of said flow restrictor; and
a valve downstream from said pressure sensor having at least two operative modes, a first mode wherein said solution is allowed to pass through said valve and a second mode wherein said solution cannot pass through said valve.

2. The system of claim 1, wherein said solution is a lavage solution.

3. The system of claim 1, wherein said valve is actuatable between said first mode and said second mode to regulate said flow of said solution.

4. The system of claim 1, wherein said flow restrictor is a capillary tube.

5. The system of claim 1, wherein said valve is a bi-stable latching valve.

6. A system for delivering a solution inside the blood-brain barrier in the brain of a patient, said system comprising:
a reservoir, implanted in said patient, containing said solution, wherein said reservoir is positively pressurized;
a flow restrictor downstream from said reservoir, in fluid communication with said reservoir; and
an infusion cannula with distal and proximal ends, wherein said distal end of said infusion cannula is located within said blood-brain barrier to deliver said solution and said proximal end of said infusion cannula is in fluid communication with said flow restrictor.

7. The system of claim 6, wherein said solution is a lavage solution.

8. The system of claim 6, further comprising a valve located between and in fluid communication with said flow restrictor and said proximal end, said valve having at least two modes, a first mode wherein said solution is allowed to pass through said valve and a second mode wherein said solution cannot pass.

9. The system of claim 8, further comprising a pressure sensor located between and in fluid communication with said flow restrictor and said valve, said sensor adapted to measure fluid pressure at said output of said flow restrictor.

10. The system of claim 8, wherein said valve is adapted to be modulated between said first mode and said second mode to regulate said flow of said solution.

11. A system for delivering a solution inside the blood-brain barrier in the brain of a patient and shunting cerebrospinal fluid away from said brain, comprising:
an infusion system capable of supplying said solution in said brain; and
a cerebrospinal shunting system capable of diverting the flow of cerebrospinal fluid away from said brain.

12. The system of claim 11, wherein said infusion system comprises:
a reservoir, implanted in said patient, containing said solution, wherein said reservoir is positively pressurized;
a flow restrictor downstream from said reservoir, in fluid communication with said reservoir; and
an infusion cannula with distal and proximal ends, wherein said distal end of said infusion cannula is located within said blood-brain barrier to deliver said solution and said proximal end of said infusion cannula is in fluid communication with said flow restrictor.

13. The system of claim 12, further comprising a valve located between, and in fluid communication with, said flow restrictor and said proximal end, having at least two modes, a first mode wherein said solution is allowed to pass through said valve and a second mode wherein said solution cannot pass.

14. The system of claim 11, wherein said solution is a lavage solution.

15. The system of claim 13, further comprising a pressure sensor located between, and in fluid communication with, said flow restrictor and said valve, said sensor adapted to measure fluid pressure at said output of said flow restrictor.

16. The system of claim 13, wherein said valve is adapted to be modulated between said first mode and said second mode to regulate said flow of said solution.

17. The system of claim 11, further comprising an implantable controller adapted to be in fluid communication with said cerebrospinal fluid and having first and second drainage paths, wherein said controller directs the flow of said cerebrospinal fluid into said first or second drainage paths in response to the inclination of said individual.

18. The system of claim 17, wherein said first drainage path is a supine flow path, and wherein said controller directs the flow of said fluid into said supine flow path in response to a supine or substantially supine position.

19. The system of claim 17, wherein said second drainage path is an upright flow path, and wherein said controller directs the flow of said fluid into said upright flow path in response to a vertical or substantially vertical position.

20. The system of claim 17, further comprising an inclination sensor for sensing the inclination of said individual, and wherein said controller is responsive to said inclination sensor.

21. The system of claim 17, further comprising a bi-stable latching valve, and wherein said controller directs the flow of said fluid by actuating said latching valve to allow for fluid communication with said first or said second drainage paths.

22. The system of claim 18, wherein said supine flow path comprises a passive low resistance flow path.

23. The system of claim 17, further comprising a programmable variable check valve in said second flow path, wherein the cracking pressure of said check valve is modified based on the inclination angle of said individual.

24. The system of claim 23, wherein said cracking pressure is continually modified to maintain a relatively stable intraventricular pressure for a range of inclination angles.

25. The system of claim 17, wherein said controller implanted in said individual further comprises:
an inlet connection;
an outlet connection spaced from said inlet connection;
an inlet cannula with distal and proximal ends, wherein said distal end of said inlet cannula is located near the ventricle of the brain and said proximal end of said inlet cannula is connected to said inlet connection of said controller; and
an outlet cannula with distal and proximal ends, wherein the location of said distal end of said outlet cannula is the peritoneal space, and said proximal end of said outlet cannula is connected to said outlet connection of said controller.

26. A method for regulating the flow of an active or inactive ingredient solution from a reservoir, comprising:
providing a flow restrictor downstream from said reservoir, in fluid communication with said reservoir;
providing a pressure sensor downstream from said flow restrictor, said sensor capable of measuring fluid pressure at the output of said flow restrictor;
providing a valve downstream from said pressure sensor having at least two modes, a first mode wherein the valve allows said active or inactive ingredient solution to pass through said valve and a second mode wherein said active or inactive ingredient solutoncannot pass;
calculating a resistance constant of said active or inactive ingredient solution when passing through said flow restrictor; and
determining the rate of said flow by dividing the pressure differential between the input and output of said flow restrictor by said resistance constant, whereby said pressure differential is calculated by calculating the difference between the measured pressure when said valve is at said second setting and the measured pressure when said valve is at said first setting.

27. The method of claim 26, wherein said valve is actuated between said first mode and said second mode to regulate said flow of said active or inactive ingredient solution.

28. A method for delivering an active or inactive ingredient solution inside the blood-brain barrier in the brain of a patient comprising:
implanting a reservoir in said patient, containing said active or inactive ingredient solution, wherein said reservoir is positively pressurized;
implanting a flow restrictor downstream from said reservoir, in fluid communication with said reservoir; and
implanting an infusion cannula with a distal and proximal end, wherein said distal end of said infusion cannula is located within said blood-brain barrier to deliver said active or inactive ingredient solution and said proximal end of said inlet cannula is in fluid communication with said flow restrictor.

29. The method of claim 28, further comprising providing a valve between and in fluid communication with said flow restrictor and said proximal end, having at least two modes, a first mode wherein the valve allows said active or inactive ingredient solution to pass through said valve and a second mode wherein said active or inactive ingredient solution cannot pass.

30. The method of claim 29, further comprising providing a pressure sensor between and in fluid communication with said flow restrictor and said valve, said sensor capable of measuring fluid pressure at output of said flow restrictor.

31. The method of claim 29, wherein said valve is modulated between said first mode and said second mode to regulate said flow of said active or inactive ingredient solution.

32. The method of claim 30, further comprising calculating a resistance constant of said active or inactive ingredient solution when passing through said flow restrictor, and calculating the rate of said flow by dividing the pressure differential between the input and output of said flow restrictor by said resistance constant, said pressure differential being calculated by calculating the difference between the measured pressure when said valve is at said second mode and the measured pressure when said valve is at said first mode.

33. A method for delivering an active or inactive ingredient solution inside the blood-brain barrier in the brain of a patient and shunting cerebrospinal fluid away from said brain, comprising:
infusing said solution in said brain; and
diverting the flow of cerebrospinal fluid away from said brain.

34. The method of claim 33, wherein said infusion further comprises:
implanting a reservoir containing said active or inactive ingredient solution in said patient, wherein said reservoir is positively pressurized;
implanting a flow restrictor downstream from said reservoir, in fluid communication with said reservoir; and
implanting an infusion cannula with a distal and proximal end, wherein said distal end of said infusion cannula is located within said blood-brain barrier to deliver said solution and said proximal end of said inlet cannula is in fluid communication with said flow restrictor.

35. The method of claim 34, further comprising implanting a valve located between, and in fluid communication with, said flow restrictor and said proximal end, having at least two modes, a first mode wherein the valve allows said active or inactive ingredient solution to pass through said valve and a second mode wherein said solution cannot pass.

36. The method of claim 35, further comprising implanting a pressure sensor located between, and in fluid communication with, said flow restrictor and said valve, said sensor capable of measuring fluid pressure at output of said flow restrictor.

37. The method of claim 35, wherein said valve is modulated between said first mode and said second mode to regulate said flow of said solution.

38. The method of claim 33, further comprising calculating a resistance constant of said active or inactive ingredient solution when passing through said flow restrictor, and calculating the rate of said flow by dividing the pressure differential between the input and output of said flow restrictor by said resistance constant, said pressure differential being calculated by calculating the difference between the measured pressure when said valve is at said second mode, and measuring the measured pressure when said valve is at said first mode.

39. The method of claim 33, further comprising implanting a controller adapted to be in fluid communication with said cerebrospinal fluid and having first and second drainage paths, wherein said controller directs the flow of said cerebrospinal fluid into said first or second drainage paths in response to the inclination of said individual.

40. The method of claim 39, wherein said first drainage path is a supine flow path, and wherein said controller directs the flow of said fluid into said supine flow path when said individual's inclination is supine or substantially supine.

41. The method of claim 39, wherein said second drainage path is an upright flow path, and wherein said controller directs the flow of said fluid into said upright flow path when said individual's inclination is vertical or greater than substantially supine.

42. The method of claim 39, further comprising implanting an inclination sensor for sensing the inclination of said individual, and wherein said controller is responsive to said inclination sensor.

43. The method of claim 39, further comprising implanting a bi-stable latching valve, and wherein said controller directs the flow of said fluid by actuating said latching valve to allow for fluid communication with said first or said second drainage paths.

44. The method of claim 40, wherein said supine flow path comprises a passive low resistance flow path.

45. The method of claim 39, further comprising implanting a programmable variable check valve in said second flow path, wherein the cracking pressure of said check valve is modified based on the inclination angle of said individual.

46. The method of claim 46, wherein said cracking pressure is continually modified to maintain a relatively stable intraventricular pressure for a range of inclination angles.

47. The method of claim 39, wherein said controller implanted in said individual further comprises:
an inlet connection;
an outlet connection spaced from said inlet connection;
an inlet cannula with a distal and proximal end, wherein said proximal end of said inlet cannula is located near the ventricle of the brain and said distal end of said inlet cannula is connected to said inlet connection of said controller; and
an outlet cannula with a distal and proximal end, wherein the location of said distal end of said outlet cannula is the peritoneal space, and said proximal end of said outlet cannula is connected to said outlet connection of said controller.
